(19) United States
(12) Patent Application Publication
(10) Pub. No.: US 2025/0014695 A1
(43) Pub. Date: Jan. 9, 2025

(54) MULTI-MODAL PATIENT REPRESENTATION

(71) Applicant: Hoffmann-La Roche Inc., Little Falls, NJ (US)

(72) Inventors: Gunther JANSEN, Lörrach (DE); Marius René GARMHAUSEN, Grenzach-Wyhlen (DE); Benjamin TORBEN-NIELSEN, Basel (CH); Otto Eric FAJARDO BENAVIDES, Birsfelden (CH); Phil Pascal ARNOLD, Basel (CH)

(21) Appl. No.: 18/888,077

(22) Filed: Sep. 17, 2024

Related U.S. Application Data
(63) Continuation of application No. PCT/US2023/015532, filed on Mar. 17, 2023.
(60) Provisional application No. 63/321,602, filed on Mar. 18, 2022.

(51) Int. Cl. G16H 10/60; A61B 5/00; G16H 50/20

(52) U.S. Cl. CPC G16H 10/60; G16H 50/20; A61B 5/7267

(57) ABSTRACT

A method implemented by one or more computer device includes accessing a set of medical data associated with a patient, in which the set of medical data includes a plurality of modalities of medical data. The method includes inputting one or more one of the plurality of modalities of medical data into a first machine-learning model trained to generate a first vector representation and inputting another one of the plurality of modalities of medical data into a second machine-learning model trained to generate a second vector representation. The method includes generating a combined vector representation based on the first vector representation and the second vector representation, and storing the combined vector representation to a database associated with the one or more computing devices.

### CROSS-REFERENCE TO RELATED APPLICATION

This application is a continuation of International Application No. PCT/US023/015532, filed on Mar. 17, 2023, which claims priority to and the benefit of U.S. Provisional Application No. 63/321,602 filed Mar. 18, 2022, the entire content of which is incorporated herein by reference.

### TECHNICAL FIELD

This application relates generally to personalized healthcare, and, more particularly, to a multi-modal patient representation that may be used for implementing personalized healthcare.

### BACKGROUND

Personalized healthcare (“PHC”) applications may generally rely upon a wide array of biological characteristics of a person or other patient data that may be associated with the person or immediate relatives of the person. Such data may include a number of different modalities, due to data being of different types and/or being obtained from different sources. When dealing with multi-modal data, it may be extremely challenging to analyze the data in a uniform and standardized manner, due to myriad variations in the data. For example, a set of multi-modal data may include data for a patient obtained from multiple data sources that capture the same general type of data, such as, for example, whole slide images (WSI), but wherein the data differs (e.g., based on the machine used by one facility to digitize the WSI versus another facility). In another example, a set of multi-modal data may include data of multiple types obtained for a patient from a single data source, such as, for example, laboratory testing data, WSI, and various omics data collected for a single patient participating in a single clinical trial. Within the scope of even just a single clinical trial, more and more various data types are being measured, analyzed, and stored as disaggregated data. In many instances, this may be complicated due to, for example, the disparate data types measuring different aspects of a patient's health, the disparate data types being measured at different scales, the disparate data types being highly variable in the degree of sparseness and noise, the disparate data types having varying longitudinal characteristics, or the disparate data types including non-random patterns of incompleteness.

For example, longitudinal patient data, such as patient laboratory testing data associated with disease development, may often take the form of either data collected over short periods of time with a higher volume of information (e.g., during a patient emergency room (“ER”) visit or hospital admittance) or data collected over longer periods of time with a lower density of information (e.g., sparseness of data or “missingness” of data). For example, for certain medical maladies (e.g., cancers, neurodegenerative diseases, and so forth), the disease development stages may span over a long and sporadic time period. For example, a patient may have patient laboratory testing data that extend back years or even decades before an actual diagnosis is ascertained. Thus, the often longitudinal nature of patient laboratory testing data may lead to the patient laboratory testing data having large, unevenly spaced measurement intervals that do not necessarily correspond to the intervals at which other types of data were collected for the patient.

Similarly, electronic health records (“EHRs”) associated with a patient may be challenging to correlate with various genomics data, proteomics data, transcriptomics data, metabolomics data, radiomics data, toxigenomics data, and/or other omics-based data deemed to be clinically significant to the patient (e.g., particular gene variants or combinations of gene variants, particular clinically significant genomic findings, particular tumor types, particular therapies, particular treatments, and so forth). Further, other medical data associated with a patient may be captured by one or more medical images, such as magnetic resonance imaging (“MRI”) images, computed tomography (“CT”) images, X-ray images, ultrasound images, positron emission tomography (“PET”) images, single-photon emission computed tomography (“SPECT”) images, digital pathology whole-slide images (“WSIs”), and so forth. While these disparate modalities of data may each individually include clinically significant information for a patient, without the ability to integrate these data into a unified representation of a patient, PHC applications, such as predicting patient survivability, patient cohort matching, or personalized drug discovery and treatment may remain elusive.

### SUMMARY

Embodiments of the present disclosure are directed toward one or more computing devices, methods, and non-transitory computer-readable media that may utilize a number of machine-learning models trained to generate a combined vector representation by combining and integrating multi-modal medical data associated with a patient, which may be further utilized to perform downstream tasks, such as one or more personalized healthcare (PHC) tasks for the patient. The present embodiments are further directed toward one or more computing devices, methods, and non-transitory computer-readable media that may encode laboratory testing data into a pictorial representation, which may be then inputted to one or more machine-learning models trained to generate a vector representation of all of the laboratory testing data associated with the patient. Indeed, in accordance with the presently disclosed embodiments, the combined vector representation may provide a unified and reduced-dimension representation of a medical of a patient, such that the combined vector representation may be utilized to perform PHC-related tasks including, for example a predicted survivability for the patient, a predicted future disease development for the patient, a predicted treatment response for the patient, a predicted diagnosis for the patient, an identified precision cohort associated with the patient, and so forth.

In this way, developers, clinicians, data scientists, and so forth may be able to use the combined vector representation for a patient and utilize it to generate a machine-learning model based predictions of clinical outcomes or other applications and insights that may be relevant in clinical and research and development (R&D) applications for the patient. In one example, the combined vector representation may better predict an overall patient survival and progression-free patient survival as compared to, for example, the various individual, disaggregated modalities of medical data. Thus, the present techniques may further increase database storage capacity and decrease processing times of the one or more computing devices, in that the combined vector representation may provide a way to store a representation of the patient's medical data with reduced dimension and magnitude. Further, by using the combined vector representation, the total number of calls to the database by the one or more computing devices during processing may be markedly reduced, thus leading to an overall decrease in processing time by the one or more computing devices.

In one or more first embodiments, one or more computing devices, methods, and non-transitory computer-readable media may access a set of medical data associated with a patient, in which the set of medical data may include a number of modalities of medical data. For example, in one embodiment, each of the number of modalities may include one of a number of data types and may be associated with a data source. In another embodiment, each of the number of modalities of medical data may include, for example, a longitudinal dataset of medical data. In certain embodiments, the number of data types may include, for example, whole slide images, radiological images, medical graph images, other medical images, genomics data, proteomics data, transcriptomics data, metabolomics data, radiomics data, toxigenomics data, multi-omics data, medication data, medical diagnostics data, medical procedures data, medical symptoms data, demographics data, patient lifestyle data, physical activity data, body mass index (BMI) data, family history data, socioeconomics data, geographic environment data, and/or other types of digital data relating to the patient. In one embodiment, the set of medical data may include, for example, one or more data sources including randomized controlled trials for medical treatment, real-world medical data, and/or patient knowledge graphs.

In certain embodiments, the one or more computing devices, methods, and non-transitory computer-readable media may then input a first one of the number of modalities of medical data into a first machine-learning model trained to generate a first vector representation of a first one of the number of modalities of medical data. In one embodiment, the first one of the number of modalities of medical data may consist of a first data type. In one embodiment, the first vector representation may include, for example, a first dimensionless value representative of a first number of datasets of the first data type. In one embodiment, the first machine-learning model may include a first convolutional autoencoder. For example, in certain embodiments, the first machine-learning model may be trained by inputting a first number of datasets to the first machine-learning model, in which the first number of datasets may correspond to the first one of the number of modalities of medical data. In certain embodiments, the first machine-learning model may be further trained by utilizing the first machine-learning model to encode the first number of datasets into the first vector representation, in which a dimension of the first vector representation is reduced with respect to a dimension of the first number of datasets.

In certain embodiments, the one or more computing devices, methods, and non-transitory computer-readable media may then input a second one of the number of modalities of medical data into a second machine-learning model trained to generate a second vector representation of the second one of the number of modalities of medical data. In one embodiment, the second one of the number of modalities of medical data may consist of a second data type. In one embodiment, the second vector representation may include a second dimensionless value representative of a second number of datasets of the second data type. In certain embodiments, the second machine-learning model may trained by input a second number of datasets to the second machine-learning model, in which the second number of datasets corresponding to the second one of the number of modalities of medical data. In certain embodiments, the second machine-learning model may be further trained by utilizing the second machine-learning model to encode the second number of datasets into the second vector representation, in which a dimension of the second vector representation is reduced with respect to a dimension of the second number of datasets. In one embodiment, the first machine-learning model was trained independently of the second machine-learning model.

In one embodiment, the first data type may include one or more whole slide images, the second data type may include genomics data, and the third data type may include laboratory testing. In one embodiment, the laboratory testing data may include one or more pictorial representations.

In certain embodiments, the one or more computing devices, methods, and non-transitory computer-readable media may generate a combined vector representation based on the first vector representation and the second vector representation. For example, in one embodiment, generating the combined vector representation may include generating a reduced-dimension dataset as compared to the set of medical data.

In certain embodiments, prior to generating the combined vector representation, the one or more computing devices, methods, and non-transitory computer-readable media may input a third one of the number of modalities of medical data into a third machine-learning model trained to generate a third vector representation of the third one of the number of modalities of medical data. In one embodiment, the third one of the number of modalities of medical data may consist of a third data type.

In certain embodiments, the one or more computing devices, methods, and non-transitory computer-readable media may then generate the combined vector representation based on the first vector representation, the second vector representation, and the third vector representation. In certain embodiments, the one or more computing devices, methods, and non-transitory computer-readable media may generate the combined vector representation by inputting the first vector representation and the second vector representation to a fourth machine-learning model, and then generating the combined vector representation by combining the first vector representation and the second vector representation utilizing the fourth machine-learning model. In one embodiment, the fourth machine-learning model may include a fully connected neural network (FCNN). In another embodiment, the fourth machine-learning model may include a deep neural network (DNN).

In certain embodiments, the one or more computing devices, methods, and non-transitory computer-readable media may then store the combined vector representation to a database associated with the one or more computing devices. In certain embodiments, the one or more computing devices, methods, and non-transitory computer-readable media may store the combined vector representation to a database associated with the one or more computing devices, methods, and non-transitory computer-readable media, and in response to receiving one or more requests for medical data associated with the patient, retrieve the combined vector representation from the database. In certain embodiments, the one or more computing devices, methods, and non-transitory computer-readable media may then perform the one or more PHC tasks for the patient based on the combined vector representation, in which the one or more PHC tasks are performed to satisfy the one or more requests.

In one embodiment, performing the one or more PHC tasks may include generating a predicted future disease development for the patient. In one embodiment, performing the one or more PHC tasks may include generating a predicted treatment response for the patient. In one embodiment, performing the one or more PHC tasks may include generating a predicted diagnosis for the patient. In one embodiment, performing the one or more PHC tasks may include identifying a precision cohort associated with the patient.

In one or more second embodiments, the one or more computing devices, methods, and non-transitory computer-readable media may access a set of medical data associated with a patient, in which the set of medical data may include longitudinal medical data. In one embodiment, the set of medical data may include a set of medical laboratory testing data. In certain embodiments, the one or more computing devices, methods, and non-transitory computer-readable media may then encode the set of medical data into a pictorial representation of the set of medical data. For example, in certain embodiments, encoding the set of medical data into the pictorial representation may include generating a number of matrices based on the medical data. In one embodiment, each of the number of matrices may include, for example, an N×N matrix of laboratory test indicators, in which an x-axis of the N×N matrix may represent time and ay-axis of the N×N matrix may represent differing medical laboratory tests.

In one embodiment, the time represented by the x-axis of the N×N matrix may include a predetermined time window for the set of medical data. For example, in one embodiment, the predetermined time window for the set of medical data may include a predetermined time window of an N number of days. In another embodiment, the time represented by the x-axis of the N×N matrix may include a configurable time window determined based on one or more attention mechanisms or activation functions utilized to determine a weight of the different medical laboratory tests. In another embodiment, the time represented by the x-axis of the N×N matrix may include one or more of a date of diagnosis, a date of an advanced diagnosis, a commencement date of a treatment regimen, a date of a patient relapse episode, a date of tumor metastasis, or a date of clinical trial randomization.

In certain embodiments, the differing medical laboratory tests represented by they-axis of the N×N matrix may include a number of laboratory test channels configured to indicate a status of the differing medical laboratory tests. In one embodiment, each of the number of laboratory test channels corresponds to a respective one of the number of matrices. For example, in certain embodiments, the number of laboratory test channels are configured to indicate the status based on whether one or more of the differing medical laboratory tests have been performed, whether a result of one or more of the differing medical laboratory tests is below normative range, whether a result of one or more of the differing medical laboratory tests is above normative range, a laboratory testing categorization for one or more of the differing medical laboratory tests, a temporal data associated with the differing medical laboratory tests, whether data derived from the one or more of the differing medical laboratory tests is normalized, or whether data derived from the one or more of the differing medical laboratory tests is featurized.

In one embodiment, one or more of the number of laboratory test channels is configured to indicate the status based on one or more color values included within a respective one of the number of matrices. In another embodiment, one or more of the number of laboratory test channels is configured to indicate the status based on one or more binary indications included within a respective one of the number of matrices. In another embodiment, one or more of the number of laboratory test channels is configured to indicate the status based on one or more numerical values included within a respective one of the number of matrices.

In certain embodiments, the one or more computing devices, methods, and non-transitory computer-readable media may then input the pictorial representation of the set of medical data into a machine-learning model trained to generate a vector representation of the set of medical data. In certain embodiments, the one or more computing devices may further evaluate the vector representation by determining a median value of survivability for a patient cohort associated with the patient, generating a predicted value of survivability for the patient based on the vector representation, comparing the median value of survivability against the predicted value of survivability to determine a difference between the median value of survivability and the predicted value of survivability, and classifying the predicted value of survivability as being greater than the median value of survivability or less than the median value of survivability based on the determined difference.

In certain embodiments, the one or more computing devices may then perform a PHC task for the patient based at least in part on the vector representation. For example, in some embodiments, performing the PHC task may include generating a predicted survivability for the patient based on the vector representation. In certain embodiments, the one or more computing devices may further input the vector representation to a perceptron machine-learning model trained to generate the PHC task. In one embodiment, the PHC task may include an indication of whether a predicted survivability for the patient is greater than or less than a median survivability. For example, in one embodiment, the predicted value of survivability may be classified as being greater than the median value of survivability or less than the median value of survivability utilizing the perceptron machine-learning model. In certain embodiments, the one or more computing devices may further determine a treatment regimen or a therapeutic regimen for the patient based on the classification of the predicted value of survivability.

### BRIEF DESCRIPTION OF THE DRAWINGS

One or more drawings included herein are in color in accordance with 37 CFR § 1.84. The color drawings are necessary to illustrate the invention. More specifically, FIGS. 3A, 3B, and 3C are images of pictorial representations of lab testing data, which color plays a predominant role in enabling one skilled in the art to understand the invention and such color drawings are the only practical medium for disclosing the subject matter to be patented.

FIG. 1 illustrates an example embodiment of a multi-modal patient representation system that may be utilized to generate a combined vector representation by combining and integrating multi-modal medical data associated with a patient.

FIG. 2 illustrates a laboratory testing encoding and patient representation system for encoding laboratory testing data into a pictorial representation and generating a vector representation based thereon.

FIG. 3A-3D illustrate one or more running example embodiments of encoded pictorial representations of patient laboratory testing data.

FIG. 4A illustrates an example embodiment of a downstream implementation and model evaluation system.

FIG. 4B illustrates an example embodiment of the downstream implementation and model evaluation system including a comparison of C-indexes.

FIG. 5A illustrates a flow diagram for generating a combined vector representation by combining and integrating multi-modal medical data associated with a patient.

FIG. 5B illustrates a flow diagram for encoding laboratory testing data into a pictorial representation, which may be then inputted to one or more machine-learning models trained to generate a vector representation of laboratory testing data associated with a patient.

FIG. 6 illustrates an example computing system.

FIG. 7 illustrates a diagram of an example artificial intelligence (AI) architecture included as part of the example computing system of FIG. 6.

### DESCRIPTION OF EXAMPLE EMBODIMENTS

Personalized healthcare (“PHC”) applications may generally rely upon a wide array of biological characteristics of a person or other patient data that may be associated with the person or immediate relatives of the person. While more and more various data types are being measured in clinical practice, for example, the various data types may often be measured, analyzed, and stored as disaggregated data. In many instances, this may be due to, for example, the various data types being measurable at different scales, the various data types including considerable sparseness and noise, the various data types having an inherent longitudinal characteristic, or the various data types including non-random patterns of incompleteness. For example, longitudinal patient data, such as patient data associated with disease development, may often take the form of either data collected over short periods of time with a higher volume of information (e.g., during a patient ER visit or hospital admittance) or data collected over longer periods of time with a lower density of information (e.g., sparseness of data or “missingness” of data). It may be useful to provide techniques to improve patient representation for PHC.

Accordingly, the present embodiments are directed toward one or more computing devices, methods, and non-transitory computer-readable media that may utilize a number of machine-learning models trained to generate a combined vector representation by combining and integrating multi-modal medical data associated with a patient, which may be further utilized to perform downstream tasks, such as one or more PHC tasks for the patient. The present embodiments are further directed toward one or more computing devices, methods, and non-transitory computer-readable media that may encode laboratory testing data into a pictorial representation, which may be then inputted to one or more machine-learning models trained to generate a vector representation of all of the laboratory testing data associated with the patient. Indeed, in accordance with the presently disclosed embodiments, the combined vector representation may provide a unified and reduced-dimension representation of a medical of a patient, such that the combined vector representation may be utilized to perform PHC-related tasks including, for example a predicted survivability for the patient, a predicted future disease development for the patient, a predicted treatment response for the patient, a predicted diagnosis for the patient, an identified precision cohort associated with the patient, and so forth.

In this way, developers, clinicians, data scientists, and so forth may be able to use the combined vector representation for a patient and utilize it to generate a machine-learning model based predictions of clinical outcomes or other applications and insights that may be relevant in clinical and research and development (R&D) applications for the patient. In one example, the combined vector representation may better predict an overall patient survival and progression-free patient survival as compared to, for example, the various individual, disaggregated modalities of medical data. Thus, the present techniques may further increase database storage capacity and decrease processing times of the one or more computing devices, in that the combined vector representation may provide a way to store a representation of the patient's medical data with reduced dimension and magnitude. Further, by using the combined vector representation, the total number of calls to the database by the one or more computing devices during processing may be markedly reduced, thus leading to an overall decrease in processing time by the one or more computing devices.

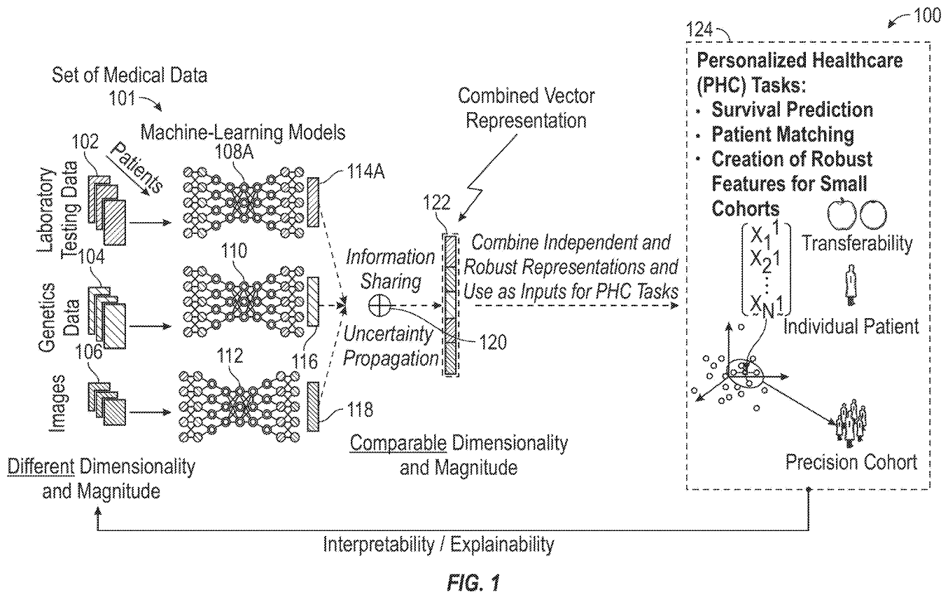

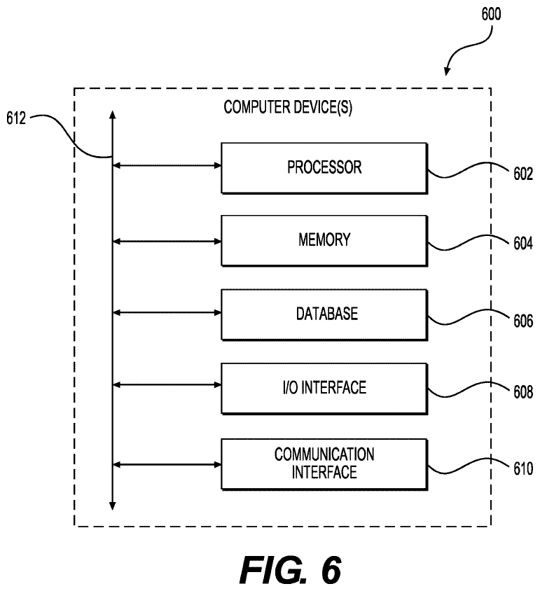

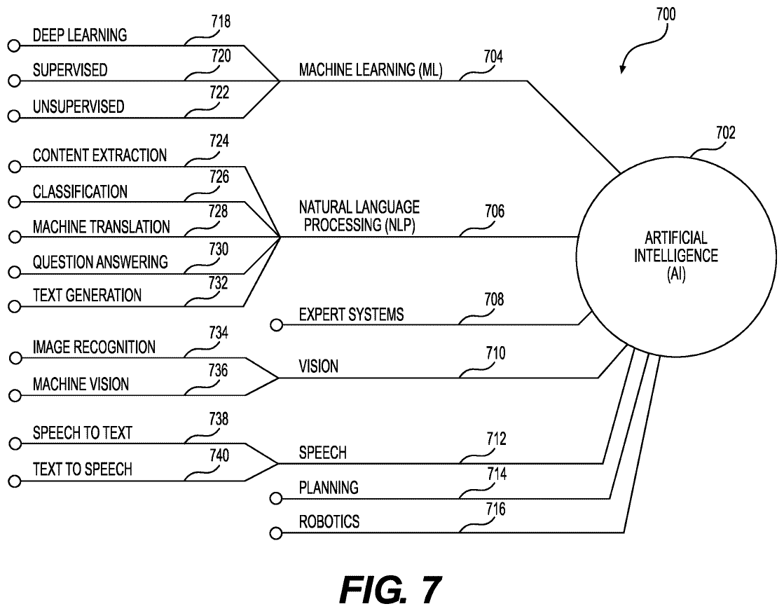

FIG. 1 illustrates an example embodiment of a multi-modal patient representation system 100 that may be utilized to generate a combined vector representation by combining and integrating multi-modal medical data associated with a patient, which may be further utilized to perform one or more PHC tasks for the patient, in accordance with the presently disclosed embodiments. In one embodiment, the multi-modal patient representation system 100 may include, for example, one or more software systems or some combination of software and hardware that may be implemented utilizing a computing system and artificial intelligence architecture to be discussed below with respect to FIGS. 6 and 7. In certain embodiments, the multi-modal patient representation system 100 may include access to a set of medical data 101. In certain embodiments, as further depicted by FIG. 1, the set of medical data 101 may include one or more modalities of patient laboratory testing data 102, one or more modalities of patient genetics data 104, and one or more modalities of patient images 106. In certain embodiments, the patient laboratory testing data 102, the patient genetics data 104, and the patient images 106 may be accessed, from one or more data sources including, for example, one or more randomized controlled trials for medical treatment, real-world medical data, one or more patient knowledge graphs, and so forth.

In one embodiment, as used herein, a randomized controlled trial (“RCT”) may refer to, for example, a study in which randomization is used to assign patients to treatments. For example, the purpose of the randomized controlled trial may be utilized to guard against any use of judgment or systematic arrangements leading to one treatment getting preferential assignment (e.g., to avoid bias), and further to provide a basis for the standard methods of statistical analysis such as significance tests. In some embodiments, as used herein, real-world medical data may refer to, for example, any data relating to patient health status and/or the delivery of health care routinely collected from a variety of sources. For example, real-world medical data may come from a number of sources, for example, electronic health records (EHRs), claims and billing activities, product and disease registries, patient-generated data including in home-use settings, data gathered from other sources that may inform on health status, such as mobile devices, and so forth. Similarly, in one embodiment, as used herein, “knowledge graphs” may represent, for example, contextual, highly connected, heterogeneous data that is organized into the form of graphs in order to contextualize search and decision-making. In the medical context, a knowledge graph may be generated based on various types of patient data.

In certain embodiments, the patient laboratory testing data 102, patient genetics data 104, and the patient images 106 may include various types of data (e.g., laboratory testing data, genetics data, medical imaging data) and various modalities of data (e.g., one or more modalities of laboratory testing data, one or more modalities of genetics data, or one or more modalities medical imaging data). In certain embodiments, the patient laboratory testing data 102 may include, for example, any patient medical data collected from a complete blood count (“CBC”) laboratory test, a prothrombin time laboratory test, a basic metabolic panel laboratory test, a comprehensive metabolic panel laboratory test, a lipid panel laboratory test, a liver panel laboratory test, a thyroid stimulating hormone laboratory test, a hemoglobin A1C laboratory test, a urinalysis laboratory test, a cultures laboratory test, and so forth.

In certain embodiments, the patient genetics data 104 may include, for example, genomics data, proteomics data, transcriptomics data, metabolomics data, radiomics data, toxigenomics data, multi-omics data, genomic findings data, targeted therapies data, targeted therapies with expected resistance data, category, genomic findings data with non-targeted therapy implications, clinical trial data, genomic findings data associated with prognostic implications category, genomic findings data associated with germline implications category, genomic findings data associated with clonal hematopoiesis (“CH”) implications, and so forth. Similarly, in certain embodiments, the patient images 106 may include, for example, whole slide images, radiological images, medical graph images, magnetic resonance imaging (“MRI”) images, computed tomography (“CT”) images, X-ray images, ultrasound images, and so forth. The patient images 106 may further include other “biological imaging”, which may include radiography, endoscopy, elastography, tactile imaging, thermography, medical photography, and nuclear medicine functional imaging techniques, such as positron emission tomography (“PET”), single-photon emission computed tomography (“SPECT”), and so forth.

In some embodiments, one or more of the patient laboratory testing data 102, the patient genetics data 104, and the patient images 106 may further include, for example, patient medication data, patient medical diagnostics data, patient medical procedures data, patient medical symptoms data, patient demographics data, patient lifestyle data, patient physical activity data, patient BMI data, patient family history data, patient socioeconomics data, patient geographic environment data, or other types of digital data relating to a patient. In certain embodiments, medical graph images may refer to, for example, any measurement and recording techniques that are primarily designed to produce electroencephalography (“EEG”), magnetoencephalography (“MEG”), electrocardiography (“ECG”), and others, and may represent other technologies that produce data susceptible to representation as a parameter graph versus time or maps that contain data about the measurement locations.

In certain embodiments, as further depicted by FIG. 1, the patient laboratory testing data 102, the patient genetics data 104, and the patient images 106 may be inputted to a respective first machine-learning model 108A, second machine-learning model 110, and third machine-learning model 112. For example, in certain embodiments, the first machine-learning model 108A, the second machine-learning model 110, and the third machine-learning model 112 may each include, for example, one or more convolutional autoencoder machine-learning models, one or more supervised autoencoder machine-learning models, a variation autoencoder (VAE), or other similar autoencoder neural network (NN) that may be trained to generate a respective first vector representation 114A, second vector representation 116, and third vector representation 118 based on the patient laboratory testing data 102, the patient genetics data 104, and the patient images 106, respectively.

For example, in certain embodiments, the first machine-learning model 108A, the second machine-learning model 110, and the third machine-learning model 112 (e.g., one or more convolutional autoencoder machine-learning models, supervised autoencoder machine-learning models, VAEs, autoencoder NNs, and so forth) may be trained to encode one or more input vectors representative of the patient laboratory testing data 102, the patient genetics data 104, and the patient images 106 into respective independent, compressed first vector representation 114A, second vector representation 116, and third vector representation 118, such that the respective independent, compressed first vector representation 114A, second vector representation 116, and third vector representation 118 may include reduced dimension and magnitude (e.g., reduced dimension and magnitude although being an exact copy of the one or more input vectors) as compared to the one or more input vectors representative of the patient laboratory testing data 102, the patient genetics data 104, and the patient images 106. Indeed, in certain embodiments, the distributed raw data and/or distributed raw features that are the patient laboratory testing data 102, the patient genetics data 104, and the patient images 106 may be transformed, for example, into one or more input vectors representative of the patient laboratory testing data 102, the patient genetics data 104, and the patient images 106 and that are suitable for being inputted into the first machine-learning model 108A, the second machine-learning model 110, and the third machine-learning model 112 (e.g., one or more convolutional autoencoder machine-learning models, supervised autoencoder machine-learning models, VAEs, autoencoder NNs, and so forth), respectively.

In certain embodiments, as further depicted by FIG. 1, the independent, compressed first vector representation 114A, the second vector representation 116, and third vector representation 118 may be then inputted into a fourth machine-learning model 120 trained to combine the independent, compressed first vector representation 114A, second vector representation 116, and third vector representation 118 into a combined vector representation 122 (this can also be called a multi-modal vector representation). For example, in certain embodiments, the fourth machine-learning model 120 may include, for example, a convolutional neural network (CNN), a fully connected neural network (FCNN), a deep neural network (DNN), or other DNN model that may be trained to generate the combined vector representation 122. In certain embodiments, the combined vector representation 122 may include, for example, a unified and reduced dimension and magnitude vector representation (e.g., a unitless vector of numerical values) of the complete medical profile of a patient. The combined vector representation 122 may be then utilized more suitably to perform PHC tasks 124 for the patient. For example, the PHC tasks 124 may include a predicted survivability for the patient, a predicted future disease development for the patient, a predicted treatment response for the patient, a predicted diagnosis for the patient, an identified precision cohort associated with the patient, and so forth.

For example, in one embodiment, the combined vector representation 122 may be utilized to identify precision cohorts across populations of patients, including for example between hospitals, countries, trials vs real world etc. Specifically, the combined vector representation 122 may be utilized to identify precision cohorts of similar patients (e.g., similar in a clinically significant manner) across a large database of patients. For example, in certain embodiments, the combined vector representation 122 may be utilized to identify precision cohorts including, for example, precision cohort having similar maladies to a patient of interest, precision cohorts having undergone successful therapies or treatment for a malady associated with the patient of interest, precision cohorts having undergone a similar disease progression as the patient of interest, similar patient cohorts that are apparently unrelated and across indications (e.g., indications of agnostic drug development), and so forth.

In certain embodiments, the combined vector representation 122 may be utilized to determine, for example, one or more similarity measures between smaller populations of patients to identify sub-cohorts of similar patients with successful therapeutic or treatment responses. In another embodiment, the combined vector representation 122 may be further utilized, for example, to perform deep patient similarity inference for accurately identifying and ranking the similarity among large or small populations of patients for PHC tasks 124 and applications. In certain embodiments, because the combined vector representation 122 may include a unified and integrated vector representation of the complete medical profile of a patient, more granular similarities to smaller precision cohorts may be identified and the results (e.g., treatment regimen responses, therapy regimen responses) from these smaller precision cohorts can be trusted as accurate having been identified utilizing the combined vector representation 122. In some embodiments, the deep patient similarity inference may further be utilized in applications, such as treatment decisions or contextualization of patient profiles at a tumor board (e.g., a group of physicians and other clinicians having various specialties that meets regularly to discuss cancer cases and exchange knowledge).

For example, in some embodiments, prior to performing the PHC tasks 124, the combined vector representation 122 may be stored to a database (e.g., database 606 as discussed below with respect to FIG. 6). In one embodiment, in response to receiving one or more requests for medical data associated with a patient (e.g., as requested by one or more clinician, medical experts, developers, data scientists, or other downstream personnel or application that may utilized the combined vector representation 122), the combined vector representation 122 may be retrieved from the database (e.g., database 606 as discussed below with respect to FIG. 6). The combined vector representation 122 may be then utilized to perform the one or more PHC tasks for the patient based on the combined vector representation 122 to satisfy the one or more requests.

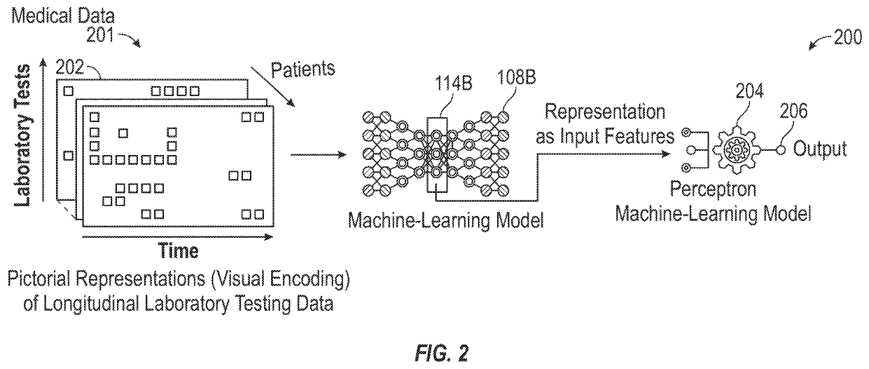

FIG. 2 illustrates a laboratory testing encoding and patient representation system 200 for encoding laboratory testing data into a pictorial representation and generating a vector representation based thereon, in accordance with the presently disclosed embodiments. In one embodiment, the laboratory testing encoding and patient representation system 200 may include, for example, one or more software systems or some combination of software and hardware that may be implemented utilizing a computing system and artificial intelligence architecture to be discussed below with respect to FIGS. 6 and 7. For example, in certain embodiments, the patient laboratory testing data 102 may include longitudinal data that may be encoded into a number of pictorial representations 202. For example, in one embodiment, the patient laboratory testing data 102 may be encoded into the number of pictorial representations 202 to compensate for instances in which the patient laboratory testing data 102 may include, for example, longitudinal data.

In such instances, because the longitudinal patient laboratory testing data 102 may include sparse data (e.g., sparse features or high instances of “missingness” of data), if the longitudinal patient laboratory testing data 102 is not first encoded into the pictorial representations 202, the machine-learning model 108B, for example, may not generate an accurate vector representation 114B representative of the longitudinal patient laboratory testing data 102. It should be further appreciated that the process of encoding the patient laboratory testing data 102 into the pictorial representations 202 may be performed before the patient laboratory testing data 102 is inputted into the machine-learning model 108B, as well as the machine-learning model 108A as discussed above with respect to FIG. 1. That is, in some embodiments, encoding the patient laboratory testing data 102 into the pictorial representations 202 as depicted by FIG. 2 may operate as a sub-process to, or in conjunction with, the process discussed above with respect to FIG. 1.

For example, for certain medical maladies (e.g., cancers, neurodegenerative diseases, and so forth), the disease development stages may span over a long and sporadic time period. For example, a patient may have patient laboratory testing data 102 that extend back years or even decades before an actual diagnosis is ascertained. Thus, the often longitudinal nature of the patient laboratory testing data 102 may lead to the patient laboratory testing data 102 having large, unevenly spaced measurement intervals. Thus, in accordance with the presently disclosed embodiments, encoding the patient laboratory testing data 102 into the pictorial representations 202 may allow patterns to be extracted from the pictorial representations 202 by the machine-learning model 108B, and may thus lead to the generation of an accurate vector representation 114B representative of the longitudinal patient laboratory testing data 102.

In certain embodiments, the pictorial representations 202 may include, for example, a number of matrices, which may each include, for example, an N×N two-dimensional matrix or an N×N×N three-dimensional matrix of laboratory test indicators. For example, in one embodiment, an x-axis of the N×N matrix may represent time and ay-axis of the N×N matrix may represent differing patient laboratory testing data 102 (e.g., various laboratory tests, such as a CBC laboratory test, a prothrombin time laboratory test, a basic metabolic panel laboratory test, a comprehensive metabolic panel laboratory test, a lipid panel laboratory test, a liver panel laboratory test, a thyroid stimulating hormone laboratory test, a hemoglobin A1C laboratory test, a urinalysis laboratory test, a cultures laboratory test, and so forth). In certain embodiments, time represented by the x-axis of each N×N matrix may include a time window, which may be a predetermined time window or a user-configurable time window.

In one embodiment, the predetermined time window or the user-configurable time window may be a time window spanning, for example, from a point in time in which a diagnosis or detection of a malady is made to a point in time afterwards (e.g., −N^{th} day to +N^{th} day). For example, in one embodiment, the predetermined time window of the patient laboratory testing data 102 may include a predetermined time window of an N number of days (e.g., a 7-day time window, a 15-day time window, 30-day time window, a 60-day time window, a 90-day time window, a 120-day time window, a 150-day time window, a 180-day time window, a 270-day time window, a 360-day time window, and so forth) for collection of the patient laboratory testing data 102. In another embodiment, the user-configurable time window may include, for example, one or more hyper-parameters determined based on one or more attention mechanisms or activation functions utilized to determine and weigh each of the various patient laboratory testing data 102.

In certain embodiments, the time represented by the x-axis of each matrix (e.g., 2D matrix or 3D matrix) may include one or more of a date of diagnosis, a date of an advanced diagnosis, a commencement date of a treatment regimen, a date of a patient relapse episode, a date of tumor metastasis, or a date of clinical trial randomization. In certain embodiments, the patient laboratory testing data 102 represented by they-axis of the each matrix (e.g., 2D matrix or 3D matrix) may include a number of laboratory test channels utilized to indicate a status of the differing laboratory testing data 102. In one embodiment, each of the number of laboratory test channels may correspond to a respective one of the number of matrices (e.g., 2D matrices or 3D matrices).

For example, in certain embodiments, the number of laboratory test channels may be utilized to indicate the status based on whether one or more of the differing medical laboratory tests (e.g., CBC laboratory tests, prothrombin time laboratory tests, basic metabolic panel laboratory tests, comprehensive metabolic panel laboratory tests, lipid panel laboratory tests, liver panel laboratory tests, thyroid stimulating hormone laboratory tests, hemoglobin A1C laboratory tests, urinalysis laboratory tests, cultures laboratory tests, and so forth) having been performed, whether a result of one or more of the differing patient laboratory tests is below normative range, whether a result of one or more of the differing medical laboratory tests is above normative range, a laboratory testing categorization for one or more of the differing laboratory tests, a temporal data associated with the differing medical laboratory tests, whether data derived from the one or more of the differing medical laboratory tests is normalized, or whether data derived from the one or more of the differing laboratory tests is featurized, and so forth.

For example, in one embodiment, one or more of the number of laboratory test channels may be utilized to indicate the status based on one or more color values included within a respective one of the number of matrices, one or more binary indications included within a respective one of the number of matrices, one or more numerical values included within a respective one of the number of matrices. For example, in some embodiments, for each of the number of laboratory test channels, a value-based indication (e.g., color-scaled to represent values from 0 to 1, one or more raw laboratory data values, normalized version of raw laboratory data, differing colors for laboratory test type categorizations, blank spaces for a particular laboratory test that has not been performed, other temporal data for how many visits, procedures, treatments, or prescription orders, and so forth) may be included within one or more cells of the matrices (e.g., 2D matrices or 3D matrices).

For example, as will be discussed in further detail below, in some embodiments, a color or shade of the number of laboratory test channels may respectively represent the status of a particular laboratory test, namely whether a particular laboratory test has been performed; if performed, whether the result is above a normative range; if performed, whether the result is below a normative range; if not performed, then the shade or color may be the same as the background color or shade (e.g., indicative of a blank space) representative of a particular laboratory test has not been performed. Specifically, in some embodiments, a blank space or other color or shade the same as the background color or shade may be processed, for example, by the machine-learning model 108B as “missingness” indicators (e.g., indicating that this lack of data for a particular point in time and/or laboratory test is itself a data point to be taken into account by the machine-learning model 108B). In one or more embodiments, one or more attention mechanisms or activation levels may be associated with the cells of the matrices (e.g., 2D matrices or 3D matrices) to determine, for example, based on the quantity or quality of data for a particular test channel whether to consider particular laboratory data test channels and/or the manner in which to weigh those particular laboratory test channels.

In certain embodiments, the pictorial representations 202 may be inputted into the machine-learning model 108B (e.g., supervised convolutional autoencoder) that may be trained to generate a laboratory testing data vector representation 114B. In certain embodiments, as further depicted by FIG. 2, the laboratory testing data vector representation 114B may be evaluated by, for example, utilizing the laboratory testing data vector representation 114B as an input vector to a machine-learning model 204. For example, in one embodiment, the machine-learning model 204 may include, for example, perceptron or other binary classifier model that may be utilized to generate an output 206 corresponding to a prediction of a survivability of the patient to which the laboratory testing data vector representation 114B corresponds. In some embodiments, the output 206 corresponding to the prediction of a survivability of the patient to which the laboratory testing data vector representation 114B corresponds may be a performance of one or more of the PHC tasks 124, which corresponds to the prediction of a survivability of the patient.

For example, in certain embodiments, evaluating the laboratory testing data vector representation 114B may include, for example, determining a median value of survivability for a patient cohort associated with the patient, generating a predicted value of survivability for the patient based on the laboratory testing data vector representation 114B, comparing the median value of survivability against the predicted value of survivability to determine a difference between the median value of survivability and the predicted value of survivability, and utilizing the machine-learning model 204 to classify the predicted value of survivability as being greater than, equal to, or less than the median value of survivability based on the determined difference.

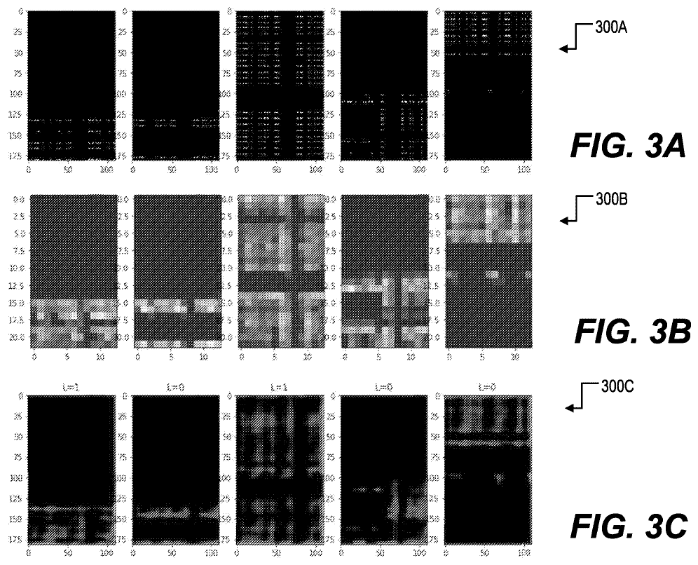

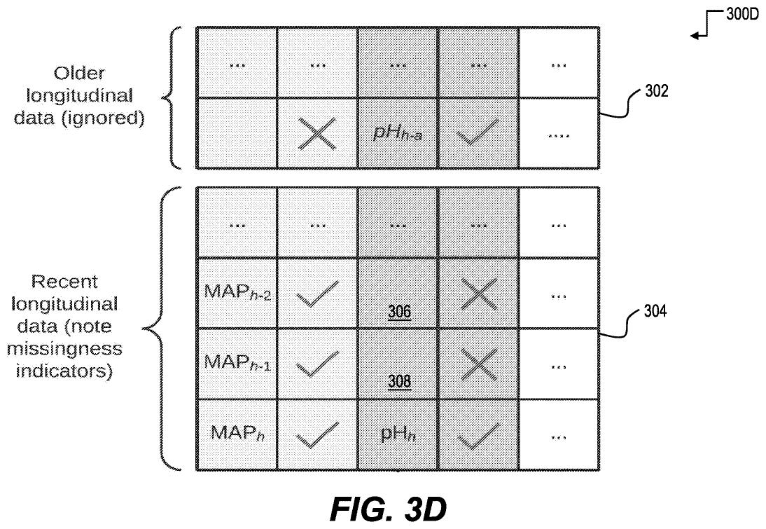

FIGS. 3A-3D illustrate one or more running example diagrams 300A-300D of encoded pictorial representations of patient laboratory testing data, in accordance with the presently disclosed embodiments. Specifically, FIGS. 3A and 3B may illustrate a real-world visual example of the encoding the patient laboratory testing data 102 (corresponding to diagram 300A of FIG. 3A) into the pictorial representations 202 (corresponding to diagram 300B of FIG. 3B), as generally discussed above with respect to FIG. 2. Similarly, FIG. 3C illustrates a decoding of the pictorial representations 202 (corresponding to diagram 300B of FIG. 3B) into another visual representation (corresponding to diagram 300C of FIG. 3C) to illustrate that the patient laboratory testing data 102 may be reconstructed from the pictorial representations 202. For example, as depicted, the diagram 300A depicts one or more matrices (e.g., 2D matrices or 3D matrices) that include laboratory testing data. In certain embodiments, the diagram 300B of one or more pictorial representations, which may be include an encoding or one or more transformations of the patient laboratory testing data 102 depicted by the one or more matrices (e.g., 2D matrices or 3D matrices).

As further depicted, the diagram 300B of the one or more pictorial representations may include, for example, varying shades (e.g., varying colors) utilized to indicate a status, a weight, a result, a temporal parameter, or other data that may be associated with the patient laboratory testing data 102. In certain embodiments, each color or shade may respectively represent the status of a particular laboratory test, namely whether a particular laboratory test has been performed; if performed, whether the result is above a normative range; if performed, whether the result is below a normative range; if not performed, then the shade or color may be the same as the background color or shade (e.g., indicative of a blank space) representative of a particular laboratory test has not been performed.

In accordance with the presently disclosed embodiments, it should be appreciated that blank spaces within the diagram 300A of one or more matrices (e.g., 2D matrices or 3D matrices) and/or corresponding pixels within the diagram 300B of one or more pictorial representations that are colored or shaded consistent with the background color or shade may represent, for example, that a particular laboratory test corresponding to those particular spaces has not been performed. The diagram 300C depicts one or more decoded representations illustrating, for example, a decoding or an inverse transformation of the diagram 300B of the one or more pictorial representations depicted by the diagram 300C. Thus, as may be perceived by comparison between the diagram 300B of one or more pictorial representations and the diagram 300C of one or more decoded representations, the one or more decoded representations of the diagram 300C is visually similar to the one or more pictorial representations of the diagram 300B.

As depicted by the diagram 300D, the representation 302 includes a previous representation in which one or more blank spaces represent data to be ignored, for example, during processing or analysis. In contrast, the pictorial representation 304 includes a number of missingness indicators or values representing, for example, that a particular laboratory test has not been performed or other material data that may be associated with a patient. For example, as previously discussed above with respect to FIG. 2, the patient laboratory testing data 102 may be sparse, in that not every patient may undergo the same testing or at the same time. Furthermore, depending on the current health of a patient, multiple physicians or other clinicians may order that different laboratory tests be performed. This may lead to interpatient differences in the availability of different laboratory tests. Still further, the frequency of particular laboratory tests may differ from patient to patient and/or with respect to the same patient over time. For example, if a given patient is hospitalized, has an acute malady, or experiences a particularly intractable phase of disease progression, daily laboratory tests may be performed as opposed to only sporadic laboratory testing that may be sufficient for other patients.

In particular example diagram 300D, the vertical axis of the pictorial representation 304 may depict a time dimension (e.g., in days). The differing shading within the pictorial representation 304 indicates a grouping of a particular test type and the result data if the particular test. Similarly, the checkmarks (e.g., “✓”) and crosses (e.g., “x”) within the pictorial representation 304 indicates whether a particular test has a result or measurement (e.g., “MAPₕ₋₂”=✓, “MAPₕ₋₁”=✓, “MAPₕ”=✓) or otherwise whether a particular test does not have a result or measurement (e.g., “pHₕ”=x). As further depicted by the diagram 300D, the respective blank spaces 306, 308 within the pictorial representation 304 may represent that a particular laboratory test has not been performed. For example, in some embodiments, the blank spaces 306, 308 may be processed, for example, by the machine-learning model 108B as “missingness” indicators (e.g., indicating that this lack of data for a particular point in time and/or laboratory test is itself a data point to be taken into account by the machine-learning model 108B).

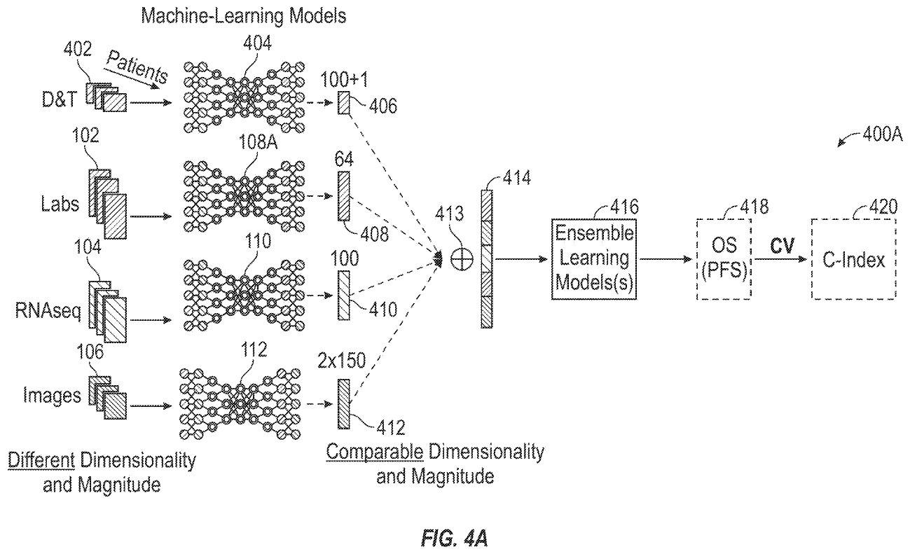

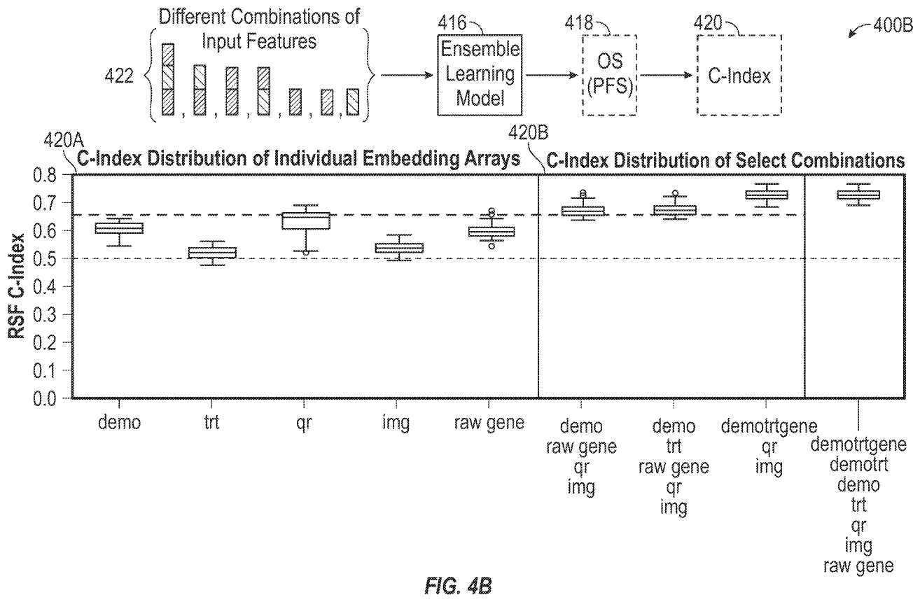

FIG. 4A illustrates an example embodiment of a downstream implementation and model evaluation system 400A, in accordance with the presently disclosed embodiments. As illustrated, various types (e.g., laboratory testing data, genetics data, medical imaging data) and modalities (e.g., one or more modalities of laboratory testing data, one or more modalities of genetics data, one or more modalities of medical imaging data) of patient data 402, 102, 104, and 106 may be inputted into respective one or more machine-learning models 404, 108A, 110, and 112 trained to generate respective independent compressed vector representations 406, 408, 410, and 412. The independent compressed vector representations 406, 408, 410, and 412 may be then inputted into an additional machine-learning model 413 that may be trained to combine the independent compressed vector representations 406, 408, 410, and 412 and to generate a combined vector representation 414. In certain embodiments, the combined vector representation 414 may be inputted into one or more ensemble learning models 416 (e.g., RSF model), which may be trained to generate an overall survival (OS) value 418. The OS value 418 may be then utilized to generate a concordance index (C-Index) 420, which may be utilized to evaluate the one or more machine-learning models 404, 108A, 110, and 112.

FIG. 4B illustrates an example embodiment of the downstream implementation and model evaluation system 400B including a comparison of C-indexes 420A and 420B, in accordance with the presently disclosed embodiments. As illustrated, differing combinations of patient vector representations 422 may be evaluated to illustrate, for example, that the combined the combined vector representation 414 performs better as compared to the independent compressed vector representations 406, 408, 410, and 412 as illustrated by C-indexes 420A and 420B, respectively. Specifically, FIG. 4B illustrates that the combined vector representation 122 generated in accordance with the presently disclosed embodiments may better predict an overall patient survival (e.g., a length of time from either the date of diagnosis or the start of treatment for a malady that patients diagnosed with the malady are still alive) and progression-free patient survival (e.g., a length of time during and after the treatment of a malady that a patient lives with the malady) as compared to, for example, the individual, independent vector representations 114A, 116, and 118 and/or combinations of pairs of the individual, independent vector representations 114A, 116, and 118. For example, in some embodiments, the individual, independent vector representations 114A, 116, and 118 may overlap and individually contribute to the overall medical profile of the patient. However, by combining the individual, independent vector representations 114A, 116, and 118 into the combined vector representation 414, a holistic and unified medical profile of a patient and the relationship of the patient to other patients may be determined. This may thus provide granular and precise information about the patient and the malady of the patient, as well as achieve better predictive performance.

For example, as an illustration of the foregoing techniques, in some embodiments, gene expression data may be combined with digital pathology image data. For example, on the one hand, the digital pathology image data may generally include, for example, a single slice taken from a tumor. Thus, the digital pathology image data may provide considerable information, for example, regarding the phenotype of the tumor, a shape of the tumor, and the microenvironment of the tumor (e.g., in the form of T-cell abundance). On the other hand, the gene expression data, which may be acquired from a piece of the bulk tumor, may provide information, for example, regarding the immune cell subtype distribution across more space of the tumor and the current state of the tumor (e.g. active tumor vs. in active tumor). Thus, in certain embodiments, by combining and aggregating the expression data with digital pathology image data, a richer and more granular understanding and characterization of the underlying biology of the tumor may be produced that otherwise not be possible utilizing the disaggregated expression data and digital pathology image data.

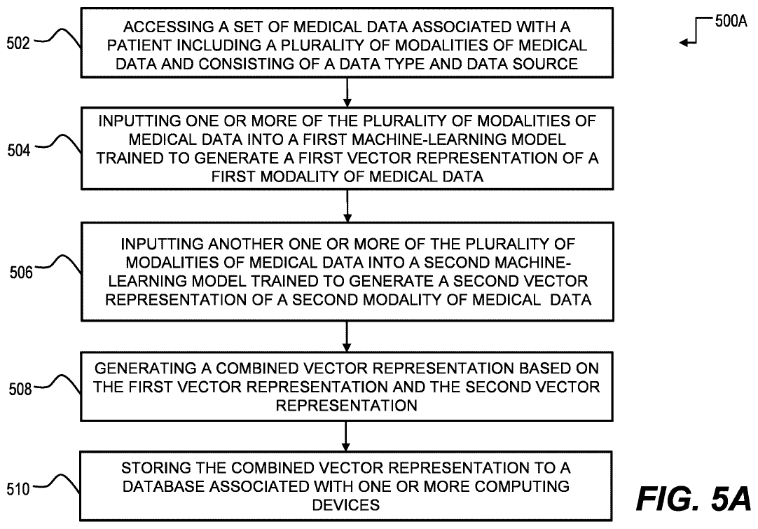

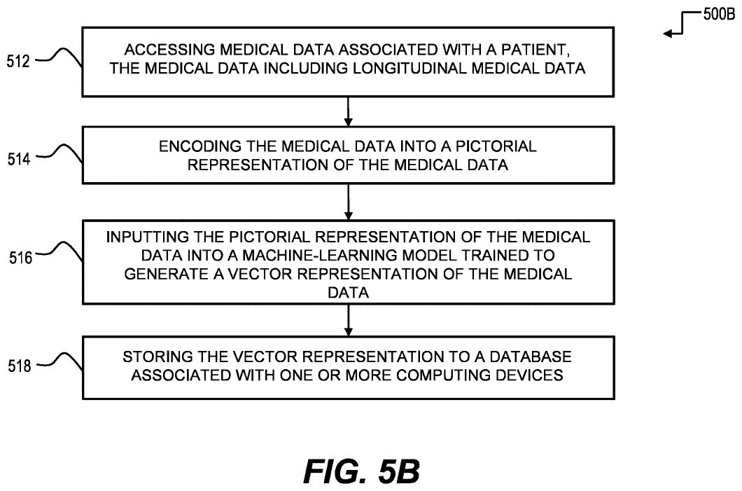

FIG. 5A illustrates a flow diagram 500A for generating a combined vector representation by combining and integrating multi-modal medical data associated with a patient, in accordance with the presently disclosed embodiments. The flow diagram 500A may be performed utilizing one or more processing devices (e.g., computing system and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various medical data and making one or more decisions based thereon), software (e.g., instructions running/executing on one or more processors), firmware (e.g., microcode), or some combination thereof.

The flow diagram 500A may begin at block 502 with one or more processing devices accessing a set of medical data associated with a patient including a plurality of modalities of medical data, in which each of the plurality of modalities consists a data type and is associated with a data source. The flow diagram 500A may then continue at block 504 with one or more processing devices inputting a first modality of medical data of the plurality of modalities of medical data into a first machine-learning model trained to generate a first vector representation of the first modality of medical data of the plurality of modalities of medical data, in which the first modality of medical data of medical data consists of a first data type. The flow diagram 500A may then continue at block 506 with one or more processing devices inputting a second modality of medical data of the plurality of modalities of medical data into a second machine-learning model trained to generate a second vector representation of the second modality of medical data, in which the second modality of medical data of medical data consists of a second data type. The flow diagram 500A may then continue at block 508 with one or more processing devices generating a combined vector representation based on the first vector representation and the second vector representation. The flow diagram 500A may then conclude at block 510 with one or more processing devices storing the combined vector representation to a database associated with the one or more computing devices.

FIG. 5B illustrates a flow diagram 500B for encoding laboratory testing data into a pictorial representation, which may be then inputted to one or more machine-learning models trained to generate a vector representation of all of the laboratory testing data associated with a patient, in accordance with the presently disclosed embodiments. The flow diagram 500B may be performed utilizing one or more processing devices (e.g., computing system and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various medical data and making one or more decisions based thereon), software (e.g., instructions running/executing on one or more processors), firmware (e.g., microcode), or some combination thereof.

The flow diagram 500B may begin at block 512 with one or more processing devices accessing medical data associated with a patient, in which the medical data comprises longitudinal medical data. The flow diagram 500B may then continue at block 514 with one or more processing devices encoding the medical data into a pictorial representation of the medical data. The flow diagram 500B may then continue at block 516 with one or more processing devices inputting the pictorial representation of the medical data into a machine-learning model trained to generate a vector representation of the medical data. The flow diagram 500B may then conclude at block 518 with one or more processing devices storing the vector representation to a database associated with one or more computing devices.

Accordingly, as generally set forth by the flow diagram 500A of FIG. 5A and the flow diagram 500B of FIG. 5B, respectively, the present embodiments are directed toward one or more computing devices that may utilize a number of machine-learning models trained to generate a combined vector representation by combining integrating multi-modal medical data associated with a patient, and that which may be further utilized to perform one or more PHC tasks for the patient. The present embodiments are further directed toward one or more computing devices that may encode laboratory testing data or one or more of the other various types of medical data and various modalities of medical data into a pictorial representation, which may be then inputted to one or more machine-learning models trained to generate a vector representation of all of the laboratory testing data associated with the patient.

Indeed, in accordance with the presently disclosed embodiments, the combined vector representation may provide a unified and reduced-dimension representation of the complete medical of a patient, such that the combined vector representation may be utilized more suitably to perform one or more PHC tasks for the patient (e.g., a predicted survivability for the patient, a predicted future disease development for the patient, a predicted treatment response for the patient, a predicted diagnosis for the patient, an identified precision cohort associated with the patient, and so forth). In this way, developers, clinicians, data scientists, and so forth may be allowed to access from a data store a singular, integrated combined vector representation indicative of a holistic and unified medical of a patient and the relationship of the patient to other patients, and then utilize that singular, integrated combined vector representation to generate machine-learning model based predictions of clinical outcomes or other applications and insights that may be relevant in clinical and R&D applications for particular patients as an optimized process.

In one example, the singular, integrated combined vector representation may better predict an overall patient survival and progression-free patient survival as compared to, for example, the individual, disaggregated various types of medical data and various modalities of medical data. Thus, the present techniques may further increase database storage capacity and decrease processing times of the one or more computing devices, in that the combined vector representation may include a reduced dimension and magnitude as compared to the disaggregated various types of medical data and various modalities of medical data. Further, by providing a singular, integrated combined vector representation, the total number of calls to the database by the one or more computing devices during processing may be markedly reduced, thus leading to an overall decrease in processing time by the one or more computing devices.

FIG. 6 illustrates an example of one or more computing device(s) 600 that may be utilized to generate a combined vector representation by combining integrating multi-modal medical data associated with a patient, which may be further utilized to perform one or more PHC tasks for the patient, in accordance with the presently disclosed embodiments. In certain embodiments, the one or more computing device(s) 600 may perform one or more steps of one or more methods described or illustrated herein. In certain embodiments, the one or more computing device(s) 600 provide functionality described or illustrated herein. In certain embodiments, software running on the one or more computing device(s) 600 performs one or more steps of one or more methods described or illustrated herein or provides functionality described or illustrated herein. Certain embodiments include one or more portions of the one or more computing device(s) 600.

This disclosure contemplates any suitable number of computing systems 600. This disclosure contemplates one or more computing device(s) 600 taking any suitable physical form. As example and not by way of limitation, one or more computing device(s) 600 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 600 may be unitary or distributed; span multiple locations; span multiple machines; span multiple data centers; or reside in a cloud, which may include one or more cloud components in one or more networks.

Where appropriate, the one or more computing device(s) 600 may perform without substantial spatial or temporal limitation one or more steps of one or more methods described or illustrated herein. As an example, and not by way of limitation, the one or more computing device(s) 600 may perform in real time or in batch mode one or more steps of one or more methods described or illustrated herein. The one or more computing device(s) 600 may perform at different times or at different locations one or more steps of one or more methods described or illustrated herein, where appropriate.

In certain embodiments, the one or more computing device(s) 600 includes a processor 602, memory 604, database 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement. In certain embodiments, processor 602 includes hardware for executing instructions, such as those making up a computer program. As an example, and not by way of limitation, to execute instructions, processor 602 may retrieve (or fetch) the instructions from an internal register, an internal cache, memory 604, or database 606; decode and execute them; and then write one or more results to an internal register, an internal cache, memory 604, or database 606. In certain embodiments, processor 602 may include one or more internal caches for data, instructions, or addresses. This disclosure contemplates processor 602 including any suitable number of any suitable internal caches, where appropriate. As an example, and not by way of limitation, processor 602 may include one or more instruction caches, one or more data caches, and one or more translation lookaside buffers (TLBs). Instructions in the instruction caches may be copies of instructions in memory 604 or database 606, and the instruction caches may speed up retrieval of those instructions by processor 602.

Data in the data caches may be copies of data in memory 604 or database 606 for instructions executing at processor 602 to operate on; the results of previous instructions executed at processor 602 for access by subsequent instructions executing at processor 602 or for writing to memory 604 or database 606; or other suitable data. The data caches may speed up read or write operations by processor 602. The TLBs may speed up virtual-address translation for processor 602. In certain embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In certain embodiments, memory 604 includes main memory for storing instructions for processor 602 to execute or data for processor 602 to operate on. As an example, and not by way of limitation, the one or more computing device(s) 600 may load instructions from database 606 or another source (such as, for example, another one or more computing device(s) 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604.

In certain embodiments, processor 602 executes only instructions in one or more internal registers or internal caches or in memory 604 (as opposed to database 606 or elsewhere) and operates only on data in one or more internal registers or internal caches or in memory 604 (as opposed to database 606 or elsewhere). One or more memory buses (which may each include an address bus and a data bus) may couple processor 602 to memory 604. Bus 612 may include one or more memory buses, as described below. In certain embodiments, one or more memory management units (MMUs) reside between processor 602 and memory 604 and facilitate accesses to memory 604 requested by processor 602. In certain embodiments, memory 604 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memory devices 604, where appropriate. Although this disclosure describes and illustrates particular memory, this disclosure contemplates any suitable memory.

In certain embodiments, database 606 includes mass storage for data or instructions. As an example, and not by way of limitation, database 606 may include a hard disk drive (HDD), a floppy disk drive, flash memory, an optical disc, a magneto-optical disc, magnetic tape, or a Universal Serial Bus (USB) drive or a combination of two or more of these. Database 606 may include removable or non-removable (or fixed) media, where appropriate. Database 606 may be internal or external to the one or more computing device(s) 600, where appropriate. In certain embodiments, database 606 is non-volatile, solid-state memory. In certain embodiments, database 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass database 606 taking any suitable physical form. Database 606 may include one or more storage control units facilitating communication between processor 602 and database 606, where appropriate. Where appropriate, database 606 may include one or more databases 606. Although this disclosure describes and illustrates particular storage, this disclosure contemplates any suitable storage.

In certain embodiments, I/O interface 608 includes hardware, software, or both, providing one or more interfaces for communication between the one or more computing device(s) 600 and one or more I/O devices. The one or more computing device(s) 600 may include one or more of these I/O devices, where appropriate. One or more of these I/O devices may enable communication between a person and the one or more computing device(s) 600. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In certain embodiments, communication interface 610 includes hardware, software, or both providing one or more interfaces for communication (such as, for example, packet-based communication) between the one or more computing device(s) 600 and one or more other computing device(s) 600 or one or more networks. As an example, and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it.

As an example, and not by way of limitation, the one or more computing device(s) 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the one or more computing device(s) 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. The one or more computing device(s) 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In certain embodiments, bus 612 includes hardware, software, or both coupling components of the one or more computing device(s) 600 to each other. As an example, and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

FIG. 7 illustrates a diagram 700 of an example artificial intelligence (AI) architecture 702 (which may be included as part of the one or more computing device(s) 600 as discussed above with respect to FIG. 6) that may be utilized to generate a combined vector representation by combining integrating multi-modal medical data associated with a patient, which may be further utilized to perform one or more PHC tasks for the patient, in accordance with the presently disclosed embodiments. In certain embodiments, the AI architecture 702 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), and/or other processing device(s) that may be suitable for processing various medical data and making one or more decisions based thereon), software (e.g., instructions running/executing on one or more processing devices), firmware (e.g., microcode), or some combination thereof.

In certain embodiments, as depicted by FIG. 7, the AI architecture 702 may include machine learning (ML) algorithms and functions 704, natural language processing (NLP) algorithms and functions 706, expert systems 708, computer-based vision algorithms and functions 710, speech recognition algorithms and functions 712, planning algorithms and functions 714, and robotics algorithms and functions 716. In certain embodiments, the ML algorithms and functions 704 may include any statistics-based algorithms that may be suitable for finding patterns across large amounts of data (e.g., “Big Data” such as genomics data, proteomics data, metabolomics data, metagenomics data, transcriptomics data, medication data, medical diagnostics data, medical procedures data, medical diagnoses data, medical symptoms data, demographics data, patient lifestyle data, physical activity data, family history data, socioeconomics data, geographic environment data, and so forth). For example, in certain embodiments, the ML algorithms and functions 704 may include deep learning algorithms 718, supervised learning algorithms 720, and unsupervised learning algorithms 722.

In certain embodiments, the deep learning algorithms 718 may include any artificial neural networks (ANNs) that may be utilized to learn deep levels of representations and abstractions from large amounts of data. For example, the deep learning algorithms 718 may include ANNs, such as a perceptron, a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning algorithms 720 may include any algorithms that may be utilized to apply, for example, what has been learned in the past to new data using labeled examples for predicting future events. For example, starting from the analysis of a known training data set, the supervised learning algorithms 720 may produce an inferred function to make predictions about the output values. The supervised learning algorithms 600 may also compare its output with the correct and intended output and find errors in order to modify the supervised learning algorithms 720 accordingly. On the other hand, the unsupervised learning algorithms 722 may include any algorithms that may applied, for example, when the data used to train the unsupervised learning algorithms 722 are neither classified nor labeled. For example, the unsupervised learning algorithms 722 may study and analyze how systems may infer a function to describe a hidden structure from unlabeled data.

In certain embodiments, the NLP algorithms and functions 706 may include any algorithms or functions that may be suitable for automatically manipulating natural language, such as speech and/or text. For example, in some embodiments, the NLP algorithms and functions 706 may include content extraction algorithms or functions 724, classification algorithms or functions 726, machine translation algorithms or functions 728, question answering (QA) algorithms or functions 730, and text generation algorithms or functions 732. In certain embodiments, the content extraction algorithms or functions 724 may include a means for extracting text or images from electronic documents (e.g., webpages, text editor documents, and so forth) to be utilized, for example, in other applications.

In certain embodiments, the classification algorithms or functions 726 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation algorithms or functions 728 may include any algorithms or functions that may be suitable for automatically converting source text in one language, for example, into text in another language. The QA algorithms or functions 730 may include any algorithms or functions that may be suitable for automatically answering questions posed by humans in, for example, a natural language, such as that performed by voice-controlled personal assistant devices. The text generation algorithms or functions 732 may include any algorithms or functions that may be suitable for automatically generating natural language texts.

In certain embodiments, the expert systems 708 may include any algorithms or functions that may be suitable for simulating the judgment and behavior of a human or an organization that has expert knowledge and experience in a particular field (e.g., stock trading, medicine, sports statistics, and so forth). The computer-based vision algorithms and functions 710 may include any algorithms or functions that may be suitable for automatically extracting information from images (e.g., photo images, video images). For example, the computer-based vision algorithms and functions 710 may include image recognition algorithms 734 and machine vision algorithms 736. The image recognition algorithms 734 may include any algorithms that may be suitable for automatically identifying and/or classifying objects, places, people, and so forth that may be included in, for example, one or more image frames or other displayed data. The machine vision algorithms 736 may include any algorithms that may be suitable for allowing computers to “see”, or, for example, to rely on image sensors cameras with specialized optics to acquire images for processing, analyzing, and/or measuring various data characteristics for decision making purposes.

In certain embodiments, the speech recognition algorithms and functions 712 may include any algorithms or functions that may be suitable for recognizing and translating spoken language into text, such as through automatic speech recognition (ASR), computer speech recognition, speech-to-text (STT) 738, or text-to-speech (TTS) 740 in order for the computing to communicate via speech with one or more users, for example. In certain embodiments, the planning algorithms and functions 714 may include any algorithms or functions that may be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics algorithms and functions 716 may include any algorithms, functions, or systems that may enable one or more devices to replicate human behavior through, for example, motions, gestures, performance tasks, decision-making, emotions, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, may be claimed in another claim category, e.g. system, as well. The dependencies or references back in the attached claims are chosen for formal reasons only. However, any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed comprises not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

### EMBODIMENTS

Among the provided embodiments are:

1. A method, comprising, by one or more computing devices:
accessing a set of medical data associated with a patient, wherein the set of medical data includes a plurality of modalities of medical data, wherein each of the modalities consists of one data type and is associated with one data source;
inputting one or more of the modalities each having a data type of laboratory testing data into a first machine-learning model trained to generate a first vector representation;
inputting another one of the modalities of medical data into a second machine-learning model trained to generate a second vector representation of the second modality of medical data, wherein the second modality of medical data consists of a second data type;
generating a combined vector representation based on the first vector representation and the second vector representation; and
storing the combined vector representation to a database associated with the one or more computing devices.

2. The method of Embodiment 1, wherein a data type consists of whole slide images, radiological images, medical graph images, other medical images, genomics data, proteomics data, transcriptomics data, metabolomics data, radiomics data, toxigenomics data, multi-omics data, medication data, medical diagnostics data, medical procedures data, medical symptoms data, demographics data, patient lifestyle data, physical activity data, body mass index (BMI) data, family history data, socioeconomics data, geographic environment data, or other types of digital data relating to the patient.

3. The method of any of Embodiments 1-2, wherein a data source consists of a randomized controlled trial for medical treatment, a provider of real-world medical data, or a provider of patient knowledge graphs.

4. The method of any of Embodiments 1-3, wherein at least one of the plurality of modalities of medical data comprises a longitudinal dataset of medical data.

5. The method of any of Embodiments 1-4, wherein the first machine-learning model was trained independently of the second machine-learning model.

6. The method of any of Embodiments 1-5, wherein the first vector representation comprises a first dimensionless value representative of a first plurality of datasets of the first data type.

7. The method of any of Embodiments 1-6, wherein the first machine-learning model comprises a first convolutional autoencoder.

8. The method of any of Embodiments 1-7, wherein the first machine-learning model was trained by:
inputting a first plurality of datasets to the first machine-learning model, the first plurality of datasets corresponding to a first modality of medical data; and
utilizing the first machine-learning model to encode the first plurality of datasets into the first vector representation, wherein a dimension of the first vector representation is reduced with respect to a dimension of the first plurality of datasets.

9. The method of any of Embodiments 1-8, wherein the second vector representation comprises a second dimensionless value representative of a second plurality of datasets of the second data type.

10. The method of any of Embodiments 1-9, wherein the second machine-learning model comprises a second convolutional autoencoder.

11. The method of any of Embodiments 1-10, wherein the second machine-learning model was trained by:
inputting a second plurality of datasets to the second machine-learning model, the second plurality of datasets corresponding to the second modality of medical data; and
utilizing the second machine-learning model to encode the second plurality of datasets into the second vector representation, wherein a dimension of the second vector representation is reduced with respect to a dimension of the second plurality of datasets.

12. The method of any of Embodiments 1-11, further comprising:
prior to generating the combined vector representation, inputting a third modality of medical profile data of the plurality of modalities of medical data into a third machine-learning model trained to generate a third vector representation of the third modality of medical data, wherein the third modality of medical data consists of a third data type; and
generating the combined vector representation based on the first vector representation, the second vector representation, and the third vector representation.

13. The method of any of Embodiments 1-12, wherein the third machine-learning model comprises a supervised convolutional autoencoder.

14. The method of any of Claims 1-13, wherein:
the second data type comprises genomics data; and
the third data type comprises laboratory testing data.

15. The method of any of Embodiments 1-14, wherein the laboratory testing data comprises one or more pictorial representations.

16. The method of any of Embodiments 1-15, wherein generating the combined vector representation comprises generating a comprehensive data representation of a biomedical of the patient.

17. The method of any of Embodiments 1-16, wherein generating the combined vector representation comprises generating a reduced-dimension dataset as compared to the set of medical data.

18. The method of any of Embodiments 1-17, wherein generating the combined vector representation further comprises:
inputting the first vector representation and the second vector representation to a fourth machine-learning model; and
generating the combined vector representation by combining the first vector representation and the second vector representation utilizing the fourth machine-learning model.

19. The method of any of Embodiments 1-18, wherein the fourth machine-learning model comprises a fully connected neural network (FCNN).

20. The method of any of Embodiments 1-19, wherein the fourth machine-learning model comprises a deep neural network (DNN).

21. The method of any of Embodiments 1-20, further comprising:
in response to receiving one or more requests for medical data associated with the patient, retrieving the combined vector representation from the database; and
performing one or more personalized healthcare (PHC) tasks for the patient based on the combined vector representation, the one or more predicted clinical outcomes being generated to satisfy the one or more requests.

22. The method of any of Embodiments 1-21, wherein performing the one or more PHC tasks comprises generating a predicted survivability for the patient.

23. The method of any of Embodiments 1-22, wherein performing the one or more PHC tasks comprises generating a predicted future disease development for the patient.

24. The method of any of Embodiments 1-23, wherein performing the one or more PHC tasks comprises generating a predicted treatment response for the patient.

25. The method of any of Embodiments 1-24, wherein performing the one or more PHC tasks comprises generating a predicted diagnosis for the patient.

26. The method of any of Embodiments 1-25, wherein performing the one or more PHC tasks comprises identifying a precision cohort associated with the patient.

27. A method, comprising, by one or more computing devices:
accessing a medical data associated with a patient, wherein the medical data comprises longitudinal medical data;
encoding the medical data into a pictorial representation of the medical data;
inputting the pictorial representation of the medical data into a machine-learning model trained to generate a vector representation of the medical data; and
storing the vector representation to a database associated with the one or more computing devices.

28. The Embodiment of Claim 27, wherein the medical data comprises laboratory testing data.

29. The method of any of Embodiments 27-28, wherein encoding the medical data into the pictorial representation comprises generating a plurality of matrices based on the medical data.

30. The method of any of Embodiments 27-29, wherein each of the plurality of matrices comprises an N×N matrix of laboratory test indicators, and wherein an x-axis of the N×N matrix represents time and a y-axis of the N×N matrix represents differing medical laboratory tests.

31. The method of any of Embodiments 27-30, wherein the time represented by the x-axis of the N×N matrix includes a predetermined time window for the medical data.

32. The method of any of Embodiments 27-31, wherein the predetermined time window for the medical data comprises a predetermined time window of an N number of days.

33. The method of any of Embodiments 27-32, wherein the time represented by the x-axis of the N×N matrix includes a configurable time window determined based on one or more attention mechanisms or activation functions utilized to determine a weight of the different medical laboratory tests.

34. The method of any of Embodiments 27-33, wherein the time represented by the x-axis of the N×N matrix includes one or more of a date of diagnosis, a date of an advanced diagnosis, a commencement date of a treatment regimen, a date of a patient relapse episode, a date of tumor metastasis, or a date of clinical trial randomization.

35. The method of any of Embodiments 27-34, of wherein the differing medical laboratory tests represented by the y-axis of the N×N matrix includes a plurality of laboratory test channels configured to indicate a status of the differing medical laboratory tests.

36. The method of any of Embodiments 27-35, wherein each of the plurality of laboratory test channels corresponds to a respective one of the plurality of matrices.

37. The method of any of Embodiments 27-36, wherein the plurality of laboratory test channels are configured to indicate the status based on whether one or more of the differing medical laboratory tests have been performed, whether a result of one or more of the differing medical laboratory tests is below normative range, whether a result of one or more of the differing medical laboratory tests is above normative range, a laboratory testing categorization for one or more of the differing medical laboratory tests, a temporal data associated with the differing medical laboratory tests, whether data derived from the one or more of the differing medical laboratory tests is normalized, or whether data derived from the one or more of the differing medical laboratory tests is featurized.

38. The method of Embodiments 27-37, wherein the plurality of laboratory test channels are configured to indicate that one or more of the differing medical laboratory tests have not been performed by a blank space.

39. The method of any of Embodiments 27-38, wherein one or more of the plurality of laboratory test channels is configured to indicate the status based on one or more color values included within a respective one of the plurality of matrices.

40. The method of any of Embodiments 27-39, wherein one or more of the plurality of laboratory test channels is configured to indicate the status based on one or more binary indications included within a respective one of the plurality of matrices.

41. The method of any of Embodiments 27-40, wherein one or more of the plurality of laboratory test channels is configured to indicate the status based on one or more numerical values included within a respective one of the plurality of matrices.

42. The method of any of Embodiments 27-41, further comprising performing one or more personalized healthcare (PHC) tasks comprising generating a predicted survivability for the patient based on the vector representation.

43. The method of any of Embodiments 27-42, further comprising:
inputting the vector representation to a perceptron machine-learning model trained to generate the one or more PHC tasks; and
performing the one or more PHC tasks utilizing the perceptron machine-learning model, the one or more PHC tasks comprising an output of whether a predicted survivability for the patient is greater than or less than a median survivability.

44. The method of any of Embodiments 27-43, further comprising:
evaluating the vector representation by:
   determining a median value of survivability for a patient cohort associated with the patient;
   generating a predicted value of survivability for the patient based on the vector representation;
   comparing the median value of survivability against the predicted value of survivability to determine a difference between the median value of survivability and the predicted value of survivability; and
   classifying the predicted value of survivability as being greater than the median value of survivability or less than the median value of survivability based on the determined difference.

45. The method of any of Embodiments 27-44, further comprising determining a treatment regimen or a therapeutic regimen for the patient based on the classification of the predicted value of survivability.

46. The method of any of Embodiments 27-45, wherein the predicted value of survivability is classified as being greater than the median value of survivability or less than the median value of survivability utilizing a perceptron machine-learning model.

What is claimed is:

1. A method (500A), comprising, by one or more computing devices (600):
accessing a set of medical data (101) associated with a patient, wherein the set of medical data (101) includes a plurality of modalities of medical data (102), (104), (106), wherein each of the modalities (102), (104), (106) consists of one data type and is associated with one data source;
inputting one or more of the modalities (102) each having a data type of laboratory testing data into a first machine-learning model (108A) trained to generate a first vector representation (114A);
inputting another one of the modalities of medical data (104), (106) into a second machine-learning model (110) trained to generate a second vector representation (116) of the second modality of medical data (104), wherein the second modality of medical data (104) consists of a second data type;
generating a combined vector representation (122) based on the first vector representation (114A) and the second vector representation (116); and
storing the combined vector representation (122) to a database (606) associated with the one or more computing devices (600).

2. The method of claim 1, wherein a data type consists of whole slide images, radiological images, medical graph images, other medical images, genomics data, proteomics data, transcriptomics data, metabolomics data, radiomics data, toxigenomics data, multi-omics data, medication data, medical diagnostics data, medical procedures data, medical symptoms data, demographics data, patient lifestyle data, physical activity data, body mass index (BMI) data, family history data, socioeconomics data, geographic environment data, or another type of digital medical data relating to the patient.

3. The method of claim 1, wherein a data source consists of a randomized controlled trial for medical treatment, a provider of real-world medical data, or a provider of patient knowledge graphs.

4. The method of claim 1, wherein at least one of the plurality of modalities of medical data (102), (104), (106) comprises a longitudinal dataset of medical data.

5. The method of claim 1, wherein the first vector representation (114A) comprises a first dimensionless value representative of a first plurality of datasets of the first data type.

6. The method of claim 1, wherein the first machine-learning model (108A) was trained by:
inputting a first plurality of datasets to the first machine-learning model (108A), the first plurality of datasets corresponding to a first modality of medical data (102); and
utilizing the first machine-learning model (108A) to encode the first plurality of datasets into the first vector representation (114A), wherein a dimension of the first vector representation (114A) is reduced with respect to a dimension of the first plurality of datasets.

7. The method of claim 1, wherein the second vector representation (110) comprises a second dimensionless value representative of a second plurality of datasets of the second data type.

8. The method of claim 1, wherein the second machine-learning model (110) was trained by:
inputting a second plurality of datasets to the second machine-learning model (110), the second plurality of datasets corresponding to the second modality of medical data (104); and
utilizing the second machine-learning model (110) to encode the second plurality of datasets into the second vector representation (116), wherein a dimension of the second vector representation (116) is reduced with respect to a dimension of the second plurality of datasets.

9. The method of claim 1, further comprising:
prior to generating the combined vector representation (122), inputting a third modality of medical data (106) of the plurality of modalities of medical data (102), (104), (106) into a third machine-learning model (112) trained to generate a third vector representation (118) of the third modality of medical data (106), wherein the third modality of medical data (106) consists of a third data type; and
generating the combined vector representation (122) based on the first vector representation (114A), the second vector representation (116), and the third vector representation (118).

10. The method of claim 1, wherein generating the combined vector representation (122) comprises generating a comprehensive data representation of a biomedical of the patient.

11. The method of claim 1, wherein generating the combined vector representation (122) comprises generating a reduced-dimension dataset as compared to the set of medical data (101).

12. The method of claim 1, wherein generating the combined vector representation (122) further comprises:
inputting the first vector representation (114A) and the second vector representation (116) to a fourth machine-learning model (120); and
generating the combined vector representation (122) by combining the first vector representation (114A) and the second vector representation (116) utilizing the fourth machine-learning model (120).

13. The method of claim 1, further comprising:
in response to receiving one or more requests for medical data associated with the patient, retrieving the combined vector representation (122) from the database (606); and
performing one or more personalized healthcare (PHC) tasks (124) for the patient based on the combined vector representation (122), the one or more PHC tasks (124) being performed to satisfy the one or more requests.

14. The method of claim 13, wherein performing the one or more PHC tasks (124) comprises generating a predicted survivability for the patient, generating a predicted future disease development for the patient, generating a predicted treatment response for the patient, generating a predicted diagnosis for the patient, or identifying a precision cohort associated with the patient.

15. A system including one or more computing devices (600), comprising:
one or more non-transitory computer-readable storage media (604) including instructions; and
one or more processors (602) coupled to the one or more storage media (604), the one or more processors (602) configured to execute the instructions and cause the system to perform the method of claim 1.

16. A method, comprising, by one or more computing devices:
accessing medical data (201) associated with a patient, wherein the medical data (201) comprises longitudinal medical data;
encoding the medical data (201) into a pictorial representation (202), (300A), (300B) of the medical data (201);
inputting the pictorial representation (202), (300A), (300B) of the medical data (201) into a machine-learning model (108B) trained to generate a vector representation (114B) of the medical data (201); and
storing the vector representation (114B) to a database (606) associated with the one or more computing devices (600).

17. The method of claim 16, wherein the medical data (201) comprises laboratory testing data (102).

18. The method of claim 16, wherein encoding the medical data (201) into the pictorial representation (202), (300A), (300B) comprises generating a plurality of matrices (300A) based on the medical data (201).

19. The method of claim 16, further comprising performing one or more PHC tasks (124) comprising generating a predicted survivability for the patient, a predicted future disease development for the patient, a predicted treatment response for the patient, a predicted diagnosis for the patient, or identifying a precision cohort associated with the patient based on the vector representation (114B).

20. A system including one or more computing devices (600), comprising:
one or more non-transitory computer-readable storage media (604) including instructions; and
one or more processors (602) coupled to the one or more storage media (604), the one or more processors (602) configured to execute the instructions and cause the system to perform the method of claim 16.
